(19) United States
(12) Patent Application Publication
(10) Pub. No.: US 2008/0293059 A1
(43) Pub. Date: Nov. 27, 2008

(54) Method of Identifying Induced Variability in in Vitro Cultures

(75) Inventors: Piotr Bednarek, Warszawa (PL); Janusz Zimny, Blonie (PL)

Correspondence Address: WALKER & JOCKE, L.P.A., 231 SOUTH BROADWAY STREET, MEDINA, OH, 44256, US

(73) Assignee: INSTYTUT HODOWLI I AKLIMATYZACJI ROSLIN (PLANT BREEDING AND ACCLIMATIZATION INSTITUTE, Blonie (PL)

(21) Appl. No.: 12/097,955

(22) PCT Filed: Dec. 22, 2005
(86) PCT No.: PCT/PL2005/000083

§ 371 Date: Jun. 18, 2008

(51) Int. Cl. C12Q 1/68

(52) U.S. Cl. 435/6

(57) ABSTRACT

The subject of the present invention is a method of identifying variability induced in in vitro cultures. The subject of the present invention may be used in the production of genetically stable lines, eg. of doubled haploids (determination of their genetic-apigenetic variability level), as well as facilitating a broadly understood, selection process of plant material in in vitro culture, previously unused in practice, for cultivation purposes, as well as the possibility of designing molecular tool for the identification of epigenetic characteristics for culturing purposes.

The subject of the present invention is a method of identifying variability induced in in vitro cultures. The subject of the present invention may be used in the production of genetically stable lines, eg. of doubled haploids (determination of their genetic-epigenetic variability level), as well as facilitating a broadly understood, selection process of plant material in in vitro culture, previously unused in practice, for cultivation purposes, as well as the possibility of designing molecular tool for the identification of epigenetic characteristics for culturing purposes.

It is a well established phenomenon that, following plant regeneration from non-differentiated tissue, the resulting population of clonal individuals is not phenotypically or genetically homogeneous. Both somaclonal and gametoclonal events contribute to this tissue culture induced variation. The term somaclonal variation was introduced by Larkin and Scowcroft [Larkin and Scowcroft 1981] to describe variation arising during somatic cell culture, while gametoclonal variation represents heritable variation arising during the culture of cells of gametic origin. The genetic basis underlying somaclonal variation remains controversial [Bouman and De Klerk 2001, Bregitzer, et al. 1998, Fourré, et al. 1997, Hossain, et al. 2003, Linacero, et al. 2000, Munthali, et al. 1996] and both the mechanisms by which it is induced, and the ways in which can be detected and exploited have been widely discussed in the literature [Brar and Jain 1998, Remotti 1998]. Genetic (DNA point mutagenesis, microsatellite sequence amplification [Linacero, et al. 2000] and transposon movement (Peshke and Phillips 1991] and epigenetic (localized changes in DNA methylation state (Jaligot, et al. 2002, Jaligot, et al. 2000))) events provide a molecular route to its induction. It is generally accepted that in vitro systems that require prolonged passage through a callus or cell suspension phase are the most vulnerable to mutation (Arene, et al. 1993, Freyssinet and Freyssinet 1988, Roseland, et al. 1991, Thomas, et al. 1982), and it has therefore been suggested that the most effective means of minimizing the variation is to attempt to induce somatic embryogenesis as rapidly as possible (Gray, et al. 1995). The definition of the molecular changes underlying de novo somaclonal variation and the quantification of its frequency have been hindered by methodological difficulties (Gaj 2001), and this is reflected in the limited description of this variation (mostly at the phenotypic level) in the literature (Guzy-Wróblewska and Szarejko 2003). However, a lack of visually detectable changes cannot be taken to infer a lack of variation at the molecular level (Gaj 2001).

DNA fingerprinting technology has the potential to detect DNA changes with growing precision and efficiency. However, neither RFLP- nor RAPD-based approaches have been particularly successful in recognizing somaclonal variants (Devaux, et al. 1993). Munthali et al. (Munthali, et al. 1996) identified only three variants out of 5607 RAPD products among a set of regenerated beet plants, and a similarly low frequency was observed in Begonia (Bouman and De Klerk 2001). Somewhat better results have been achieved in studies of protoplast-derived regenerants of Lolium (Wang, et al. 1993), and from long-term wheat cell culture suspensions (Brown, et al. 1993). The AFLP technique is potentially more appropriate as a detection platform, since it both identifies a relatively large number of amplified fragments, and is more experimentally robust than RAPD, as a result of its use of a more stringent PCR regime. Despite this, relatively few reports of its use to detect variation in methylation pattern or somaclonal variation have been published to date (Cervera, et al. 2002, Portis, et al. 2003, Vendrame, et al. 1999). As an example, it was able to unequivocally discriminate between embryogenic culture lines of Carya illinoinenisis and was able to group embryos originating from a given line (Vendrame, et al. 1999).

A feature of AFLP is the flexibility of choice for the two restriction enzymes needed for the initial digestion of template DNA. Where one or both are sensitive to methylation, the loss or gain of fragments in a profile can be due either to gain/loss of restriction sites (sequence variants) or to differential methylation of particular recognition sites, as pointed out by Donini et al. (Donini, et al. 1997). Isoschizomeric comparisons, in which one treatment uses a methylation-sensitive, and the other a methylation-insensitive enzyme have been explored in Arabidopsis (Cervera, et al. 2002). This method, termed methylation sensitive amplified polymorphism (MSAP), is particularly suited to the analysis of the molecular basis of somaclonal variation. The present study describes the elaboration and application of an analytical method, which enables the detection of tissue culture-induced variation in barley (Hordeum vulgare L.) both at the nucleotide sequence and at the DNA methylation level. Our aim was further to compare the frequency and patterns of variation between two different sources of explants (immature embryos and microspores) and to evaluate the potential of the approach to quantify the contributions of methylation and sequence alteration to overall variation. We propose a method flexible enough to study different parts of the genome, and tailored to giving either a qualitative and quantitative description of tissue culture-induced variation.

Patent description U.S. Pat. No. 6,300,071 (published 2001 Oct. 9) describes a method for detecting nucleic acid methylation using AFLP(TM). The invention relates to a method for analyzing of determining the methylation pattern of a starting DNA and/or for distinguishing between methylated and non-methylated sites in the starting DNA, comprising at least (A) generating a first DNA fingerprint, containing bands corresponding to both the methylated and non-methylated sites of interest; and/or (B) generating a second DNA fingerprint, containing bands corresponding only to the methylated sites of interest; and optionally comprising (C) generating a third DNA fingerprint, containing bands corresponding only to the non-methylated sites of interest; and optionally further comprising (D) analysing the fingerprint(s) thus obtained. The fingerprints are preferably generated using AFLP, by means of a frequent cutter and a methylation sensitive rare cutter. The invention further relates to specific methods for generating the above first and second DNA fingerprint by means of AFLP, and kits for use with said methods.

Patent description WO9953100 (published 1999 Oct. 21) describes a method for finding genetic markers of somaclonal variation. The invention provides a method for obtaining molecular markers for use as a diagnostic and quality control tool to identify genomic polymorphisms that arise during the process of tissue culture of in vitro propagated plants. By using a representational difference analysis (RDA) adapted for plant genomes, a set of nucleic acid difference sequences between normal and off-type plant genomes are obtained. The invention further provides a method for isolating sets of variant sequences which are common to many naturally occurring or tissue culture-generated off-types of the same cultivar or species, in addition to variant sequences present in all off-types, regardless of the phenotypic mutation, and/or in all off-types that exhibit the same mutation. Detection of somaclonal variation by the method of the invention may present an opportunity to optimize tissue culture conditions and to optimize plant multiplication rates without producing a significant number of off-types.

Despite the above mentioned research on methods of identifying induced variability in in vitro cultures applicable in such uses as obtaining genetically equivalent lines, and which also facilitate the performance of broadly understood selection of plant material derived via in vitro cultures for cultivation purposes, as well as the possibility of producing a molecular tool for the identification of epigenetic characteristics for cultivation purposes, a need still exists to produce an effective solution for the quantitative and qualitative evaluation of variance induced in in vitro cultures and somaclonal variance.

The goal of the present invention is to provide tools which could be used into obtain a method of quantitative and qualitative estimation of induced variability in in vitro cultures as well as stably inherited changes in subsequent generative generations. The goal of the present solution is to produce genetically stable lines, including doubled haploids, to determine the level of their genetic-epigenetic variability, to facilitate the selection of plant material derived via in vitro culturing for cultivation purposes, as well as producing easily adaptable molecular tools for the identification of their epigenetic characteristics for culturing purposes. The goal is to obtain a method of quantitatively characterizing the variability induced in in vitro cultures as well as variation inherited subsequent generative generations, which would facilitate the evaluation of individual types of variability (sequence and methylation variability, divided into more specific subtypes) which would allow one to determine which of the types (subtypes) of variability is dominant, whether sequence variability is caused by mobile element migration, changes in micro- and macrosatellite regions, within gene families, etc.

The realization of such a stated goal to provide solutions to the problems described in the state of the art, in order to provide tools which could be used into obtain a method of quantitative and qualitative estimation of induced variability in in vitro cultures as well as variation inherited as a result of generative propagation, which would facilitate the selection of plant materials derived through in vitro culture for cultivation purposes, the application of said methodology to determine what percentage of variability sequence or methylation variability (methylated and non-methylated sites) was stably passed onto plant progeny, were achieved in the present invention.

The topic of the invention is a method for the identification of variability induced by the derivation method in in vitro cultures at the level of regenerant plants, as well as variation inherited as a result of generative propagation, characterized in that the method consists of the following stages:
1) preparation of a single plant, a doubled haploid, derived via direct embryogenesis from a single microspore and/or egg cell to be used in the production of donor plants through self-pollination, which are to be a control for the level of generative reproduction-caused genetic and epigenetic variability; wherein the donor plants constitute a source of explants used for in vitro cultures, where each donor plant is an ancestor of a separate set of regenerants, and each regenerant is used to obtain a set of plants which are the progeny of a given regenerant;
2) the production of regenerants derived through somatic embryogenesis, pollen androgenesis and isolated microspores, or chromosome elimination, henceforth called methods of derivation, where the regenerants serve to estimate genetic and epigenetic changes induced directly by their method of derivation;
3) production of generative progeny of regenerants, which are derived via somatic embryogenesis, pistil androgenesis and isolated microspores or chromosome elimination, wherein this progeny along with the donor plant constitute the research material for the analysis of variability inherited generatively, henceforth called somaclonal variability;
4) in donor plants and an individual plant which are controls of generative propagation, estimation of genetic and epigenetic variability induced during the generative propagation of an individual plant in two AFLP systems, using isoschizomers both sensitive and insensitive to methylation at the restriction-site and/or adjacent areas, and a representative number of pairs of selective primers identical in all AFLP analyses, where:
   a) the estimation of genetic and epigenetic variability induced during the generative propagation of an individual plant in two AFLP systems among donor plants is alternatively conducted without an individual plant, but with the use of isoschizomers both sensitive and insensitive to methylation at the restriction-site and/or adjacent areas as well as a representative number of pairs of selective primers identical in all AFLP analyses;
   b) the estimation of genetic and epigenetic variability induced during the generative propagation of an individual plant in two AFLP systems among donor plants is alternatively conducted without an individual plant, but with the use of isoschizomers both sensitive and insensitive to methylation at the restriction-site and/or adjacent areas as well as a representative number of pairs of selective primers different from those used in all AFLP analyses;
5) estimation of the repeatability of the two AFLP technique variants: repeatability control of AFLP based on the identity of several DNA samples of the same plant through the use of AFLP and isoschizomers both sensitive and insensitive to methylation at the restriction-site and/or adjacent areas as well as a representative number of pairs of selective primers identical in all AFLP analyses;
6) estimation of the repeatability of the two AFLP technique variants: repeatability control of AFLP based on the identity of several DNA samples of the same plant through the use of AFLP and isoschizomers both sensitive and insensitive to methylation at the restriction-site and/or adjacent areas as well as a representative number of pairs of selective primers using a different set of selective AFLP primers than those used in the other AFLP analyses;
7) identification of variability induced by the method of derivation of the regenerants in relation to the donor plant is performed in two AFLP variants using isoschizomers both sensitive and insensitive to methylation at the restriction-site and/or adjacent areas as well as a representative number of pairs of selective primers identical in all AFLP analyses;
8) identification of the tissue culture induced variability among regenerants in relation to the donor plant is performed in two AFLP variants using isoschizomers both sensitive and insensitive to methylation at the restriction-site and/or adjacent areas as well as a representative number of pairs of selective primers identical in all AFLP analyses;
9) the identification of somaclonal variability among regenerant progeny in relation to the donor plant is carried out using two AFLP technique variants z utilising isoschizomers both sensitive and insensitive to methylation at the restriction-site and/or adjacent areas as well as a representative number of pairs of selective primers identical in all AFLP analyses;
10) the molecular analysis of the experimental material using the AFLP technique taking into account isoschizomers both sensitive and insensitive to methylation at the restriction-site and/or adjacent areas as well as a representative number of pairs of selective primers identical in all AFLP analyses is comprised of the following stages:
   a) analyses of AFLP patterns of donor plants with respect to genetic and epigenetic variability induced during the generative propagation of an individual plant, a control of generative propagation,
   b) analyses of AFLP patterns for the control of the reproducibility of both AFLP technique variants,
   c) analyses of AFLP patterns for regenerants in comparison to donor plants or separate regenerants in relation to appropriate donor plants;
   d) analyses of AFLP patterns for donor plant, its regenerant and regenerant progeny, or distinct regenerant progeny lines along with appropriate donor and its regenerant plants;
11) identification of the 14 types of events recognized at the binary matrix level for systems sensitive and insensitive to methylation, which are the result of a comparison between the donor plant with regenerants and between donor plant with regenerant progeny simultaneously in two AFLP systems, wherein the identification of event types is carried out individually for each regenerant and as bulk for regenerant progenies and is represented as 14 binary matrices which describe each of the events independently, subdivided into methods of derivation; wherein the columns represent consecutive regenerants or bulks and rows represent consecutive AFLP fragments;
12) assignment of certain event types to a given type of variability induced the method of derivation in relation to each regenerant;
13) identification of the 14 types of events recognized at the binary matrix level for systems sensitive and insensitive to methylation, which are the result of a comparison between the donor plant with regenerant simultaneously in two AFLP systems;
14) identification of the 14 types of events recognized at the binary matrix level for systems sensitive and insensitive to methylation, which are the result of a comparison between the donor plant with regenerant progeny simultaneously in two AFLP systems, where the identification of event types is performed through the summation of the genotypes of a given summary progeny, and through the comparison of such a total AFLP profile of regenerant progeny with the donor plant profile,
15) the bulk regenerant progeny genotype expressed via an AFLP profile for systems sensitive and insensitive to methylation at the level of individual AFLP fragments has a different value from that of the donor plant in a given AFLP system, so long as a given fragment within it is polymorphic, or is totally different from this value for the donor plant; in the opposite case the values for the donor plant and bulk regenerant progeny for a given AFLP fragment in a given AFLP system are identical.
16) production of a quantitative matrix for regenerants, where the columns contain data for consecutive regenerants grouped by the method of derivation, and the rows contain the 14 types of events which may be assigned to each regenerant progeny;
17) production of a quantitative matrix for regenerant progeny, where the columns contain bulk data for regenerant progeny grouped by the method of derivation, and the rows contain the 14 types of events which may be assigned to each bulk regenerant progeny;
18) production of a quantitative matrix for events in regenerants, where this is a quantitative matrix for post-transposition regenerants, where the columns contain 14 events and the rows contain regenerants grouped by the method of derivation;
19) calculation of the total number of events based on the binary matrix for individual methods of derivation of regenerants, as well as for regenerants, where the total number of events is calculated as a product of the number of AFLP fragments identified in both AFLP systems for all pairs of selective primers multiplied by the number of regenerants derived by a given method;
20) calculation of the total number of events based on the binary matrix for individual methods of derivation of regenerants as well as for regenerant progenies in bulk, where the total number of events corresponds to the number of DNA fragments identical or different in both AFLP systems for all pairs of selective primers used in the analysis;
21) calculation of the error stemming from generative propagation is carried out based on the binary matrix of donor plants and a single plant; determination to of the number of events different from those in a single plant belonging to the donor plants, and the calculation of the total number of events for the donor plants;
22) calculation of the error stemming from the AFLP technique is carried out based on the binary matrix for DNA samples obtained from the same plant and subjected to the stages of the AFLP technique using isoschizomers sensitive and insensitive to methylation at the restriction site or its immediate surroundings as well as a representative number of selective starter pairs identical in all AFLP systems, where the total number of events and the number of polymorphic events are calculated, and the latter number is expressed as a percentage;
23) determination, based on binary matrices for regenerants and the donor plant, of groups of events describing: (a) sequence variation connected with any changes in DNA nucleotide sequences based on AFLP data from the system insensitive to methylation and (b) epigenetic variability, the de novo variability of methylation patterns, and additionally mixed variation which is sequence and methylation variability based on data for both AFLP systems;
24) determination, based on binary matrices for bulk regenerant progeny and the donor plant, of event groups describing (a) sequence variation connected with all DNA nucleotide changes based on AFLP data from the system insensitive to methylation and (b) epigenetic variability, the de novo variability of methylation patterns, and additionally mixed variation which is sequence and methylation variability, and (c) inheritance of methylation patterns as related to the presence and/or lack of methylation at the restriction site or its vicinity, based on data for both AFLP systems;
25) identification of events particularly subject to the activity of factors inducing variability in in vitro cultures;
26) sequence analysis of individual event types and/or broad sequence variability, where variability is identified using AFLP technique variants using primers directed against conservative sequences within mobile elements, repeating sequences or the sequences of conservative gene families;
27) statistical analysis of the donor plant and its regenerants as well as regenerant progeny, and possibly distinct regenerants and regenerant progenies along with appropriate donor plants, as well as AFLP reproducibility control and generative propagation control:
   a) statistical analysis of the results of AFLP using parametric analysis, Mantel's test and/or Cronbach's alpha coefficient as well as molecular variance analysis using the Fₛₜ or Phiₛₜ coefficients;
   b) determination of the error of the tissue culture induced and somaclonal variability estimates, based on the Mantel test correlation coefficient;
   c) determination of the quantitative parameters of variability induced by the method of derivation of regenerants and somaclonal variability for regenerants, individual regenerant progenies, taking into account the method of derivation and giving their statistical significance level;
   d) statistic determination of differences between individual types of events induced in in vitro cultures for regenerants as well as bulk regenerant progeny, grouped by method of derivation and supplemented by their statistical significance using quantitative matrices of events;
   e) statistical analysis of the comparison between variability induced by the method of derivation as well as somaclonal variation, determined through the comparison of the donor plant profile, and the bulk profile of regenerant progeny and their types: sequence variability and/or broad sequence variability, de novo methylation, demethylation, mixed variability, inheritance of: methylation patterns, non-methylated sites or conservative sites in regenerant progeny, grouped by method of derivation as well as a comparison by method of derivation of regenerant progeny, where quantitative matrices are used for events;
   f) statistical comparisons of regenerants, and the bulk regenerant progenies for different methods of derivation for a given regenerant progeny, where quantitative matrices of the progeny are used;
   g) statistical comparison of the results of the above mentioned points for individual regenerants and bulk regenerant progenies amongst one another;
   h) statistical or qualitative comparison of somaclonal variation between individual plants being the generative progeny of regenerants;
   i) collation of the existing results in the form of a compilation indicating quantitative characteristics of variability induced by the method of derivation determined at the regenerant level, as well as quantitative characteristics of inherited variability determined at the level of regenerant progeny.

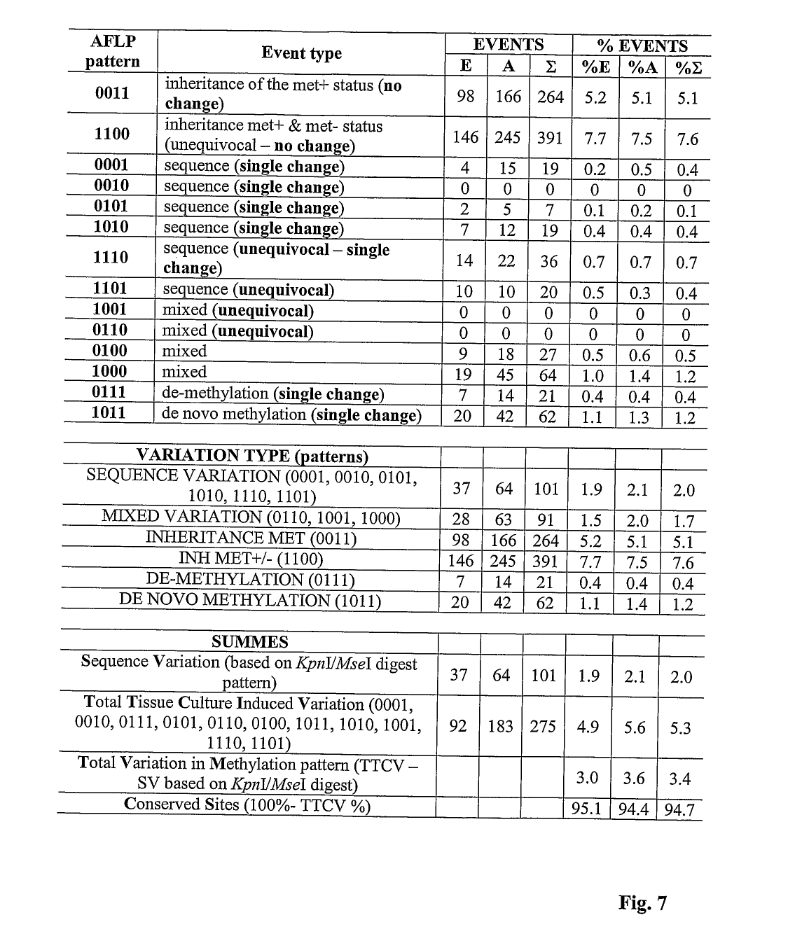

Preferentially, the identification of the 14 types of events recognized at the level of binary matrix is selected from among the events represented in FIG. 7.

Preferentially, that to derive donor plants, a single plant is used which is a doubled haploid derived from an isolated microspore, wherein it is treated as a control of the genetic and epigenetic variability of donor plants.

Preferentially, that genetic and epigenetic variability of generatively propagated material, donor plants, is determined based on AFLP profiles obtained from restriction endonuclease isoschizomers recognizing and digesting identical sequences, but differing in their sensitivity to methylation at or about the restriction site, wherein one of the isoschizomers is insensitive to methylation.

Preferentially, that in the system sensitive and insensitive to methylation no differences are observed in the AFLP profiles of donor plants, then the level of variability induced through generative propagation and identified with a given pair of selective primers is insignificant.

Preferentially, that when there are differences in AFLP profiles for donor plants between a system sensitive and a system insensitive to methylation, a percentage portion of such an event is determined, along with the background level, generative propagation error.

Preferentially, that when there are differences in AFLP profiles for experimental replicants on the same DNA samples, the AFLP technique error is estimated in order to determine experimental error.

Preferentially, the integral portion of the research material is composed of donor plants, along with a single plant, donor plant regenerants and separate regenerant progenies.

Preferentially, the donor plant along with regenerants and regenerant progeny compose the core of research material for the study of variability induced by the method of derivation or somaclonal variability, respectively.

Preferentially, that the molecular analysis is performed using the AFLP technique in two variants, using isoschizomers differing in their sensitivity to methylation.

Preferentially, that one of the isoschizomers is completely insensitive to methylation.

Preferentially, that the AFLP technique is composed of the following stages: isolation of genomic DNA, digestion of the genomic DNA with one pair of restriction endonucleases and digestion of another portion of the same DNA with another pair of restriction endonucleases, annealing of synthetic heteroduplexes to the free restriction ends, initial amplification of fragments obtained in such a manner by way of primary PCR, selective amplification, separation of the PCR products in a polyacrylamide gel, profile analysis.

Preferentially, that in the identification of PCR products an isotope marker is used which is introduced to the 5′ end of the primer complementary to the adaptor annealed to the restriction site freed by the isoschizomers.

Preferentially, that other methods of visualising AFLP fragments are used, including fluorescence.

Preferentially, the profiles of both AFLP variants for the donor plant along with its regenerants and taking into account the method of derivation and AFLP reproducibility control are performed on identical AFLP primer pairs.

Preferentially, that AFLP profile analysis and their conversion to binary matrices is based on the fact that the AFLP profiles of both restrictase systems for all pairs of selective primers are entered as a binary matrix into a spreadsheet, where the data set obtained for one pair of selective AFLP primers is placed below the previous one such that each column represents a total binary profile for a donor plant in one AFLP system, and the subsequent columns contain analogous data for progeny and controls, wherein data for the second AFLP system is recorded in subsequent columns, wherein the bands from one system are ordered like those from the previous system (in rows), and where a given band does not appear a zero is entered in the appropriate row of the spreadsheet. Preferentially, AFLP profile analysis and their conversion into binary matrices is based on the fact that the spreadsheet is filled with AFLP profiles for both restriction enzyme systems for all selective primer pairs in the form of a binary matrix, where the set of all data obtained for one selective AFLP primer pair is placed below the previous one, such that a bulk binary profile may be obtained for a donor plant per AFLP system, and consecutive columns contain analogous data for regenerant plants and controls, where analogously consecutive columns are completed with data for the second AFLP system, wherein the bands of one system are matched in rows with bands from the next system, wherein if a signal does not appear in one system, a zero value is entered in the appropriate row.

Preferentially, the AFLP profile for regenerant progeny is represented in the form of a bulk genotype per AFLP variant, sensitive and insensitive to methylation separately.

Preferentially, the generative propagation control is carried out through the summation of the number of signals (events) from a single plant, where the number of changes is calculated as a percentage of the total number of events.

Preferentially, the parametric analysis, Mantel's test correlation analysis and molecular variability analysis are performed using binary matrices of AFLP systems sensitive and insensitive to methylation for regenerants and/or regenerant progeny depending on whether the variability induced in culture or inherited by regenerant progeny are being analyzed.

Preferentially, that the factorial analysis is used as the graphical representation of the AFLP results.

Preferentially, the identification of the 14 types of events recognized at the level of AFLP pattern and assigned to variability induced by the method of derivation is performed on the basis of AFLP patterns, wherein the donor plant profile is used along with regenerant profiles in both systems, and subsequently the profile of each regenerant is compared to the donor plant profile in order to identify these events.

Preferentially, the identification of the 14 types of events recognized at the level of AFLP patterns and assigned to somaclonal variability is carried out on the basis of AFLP patterns, where the donor plant profile is used along with profiles in both AFLP systems, and the bulk profile of regenerant progeny is compared to the donor plant profile in order to identify event types, wherein it is assumed that in case of polymorphic AFLP fragments of the bulk regenerant progeny genotype, a value different than that of the donor plant is entered or a different value if it is different from that of the donor plant in all regenerant progenies for a given AFLP system.

Preferentially, when the 14 event types comprise the basis for their segregation into groups: sequence variability, methylation variability (methylation and demethylation), mixed variability and inheritance of methylation patterns, wherein it is taken into account that sequence variability is unequivocally confirmed in a system insensitive to methylation.

Preferentially, the analysis of the significance of individual variability types and events within regenerants and regenerant progeny plants, as grouped by the method of derivation, is performed based on qualitative matrices of regenerants and quantitative matrices of events describing all theoretically possible events, as well as regenerant plants and regenerant progeny.

Preferentially, the statistical analysis results for individual regenerants and bulk regenerant progenies obtained through various means are compiled into a whole; quantitative parameters are entered for variability induced by the method of derivation and of somaclonal variability for individual regenerants and bulk regenerant progenies as well as for separate regenerant progenies grouped by the method of derivation and event types along with statistical parameters.

Preferentially, those event types are identified, which are rare in culture or which do not occur at all, as well as those which are particularly subject to the activity of factors inducing variability at the level of regenerant and regenerant progeny, as well as to determine their quantitative parameters.

Preferentially, individual event types are studied at the molecular level through the sequence analysis of AFLP fragments representing individual types of events, and by the same token types of somaclonal variability.

Preferentially, one or more bulk regenerant progeny is analyzed.

Preferentially, the quantitative characteristics of an event are given for each individual bulk regenerant progeny: sequence variability, broad sequence variability, epigenetic variability of methylation patterns (de novo methylation, demethylation), stability of inherited methylated and non-methylated sites (conservative), against changes induced in cultures as well as entering quantitative differences in variability between different derivation methods of plant material.

Preferentially, statistical analysis is performed of the significance of individual event types among the regenerants, bulk regenerant progeny and individual regenerant progenies as needed, grouped by the method of derivation of the research material.

Preferentially, the somaclonal variability level of individual plants which are regenerant progeny is determined independently, using the principles used for somaclonal variability;

Preferentially, when testing hypotheses relating to the sources of variability induced by the method of derivation of regenerants and that inherited as a result of generative propagation are tested.

Preferentially, the detailed analysis is performed of the causes of sequence variability after introducing minor AFLP procedural changes, based on the selective amplification of research material using a tagged primer complementary to conservative sites of transposable elements, or to macrosatellite sequences.

Preferentially, segregation of genetic and epigenetic markers are analyzed for mapping populations derived through plant-plant crosses which are the progeny of regenerants with a known genotype, based on AFLP profiles for both AFLP systems.

Preferentially, the method is designed for deriving plants in vitro with a lowered or enhanced level of somaclonal variability through the identification of the causes of this variability.

Preferentially, the genetic and epigenetic purity of arbitrary material derived through in vitro culture is verified.

Preferentially, when the method is used in the analysis of the inheritance of epigenetic variability during sexual crossing.

Preferentially, the method is a tool for the identification of epigenetic markers linked with the utilitarian characteristics of the plant material.

Preferentially, the method is used for the identification of differences in DNA methylation patterns for various tissues of an individual plant.

The attached figures facilitate a better description of the nature of the present invention.

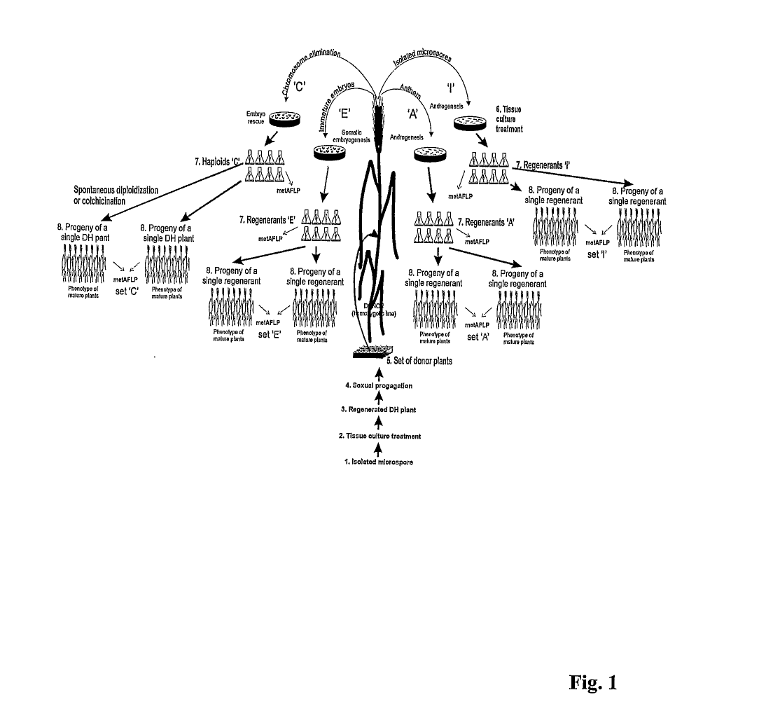

FIG. 1 represents general scheme for the evaluation of plant materials to study tissue culture induced (level 7) and somaclonal variation (level 8) due to regeneration method (level 6). Generative reproduction induced variation and purity of the initial material along with the reproducibility of the AFLP approaches are verified at 5. In the described example regenerants and donor plants were analyzed (level 7) following embryo- and androgenesis paths only.

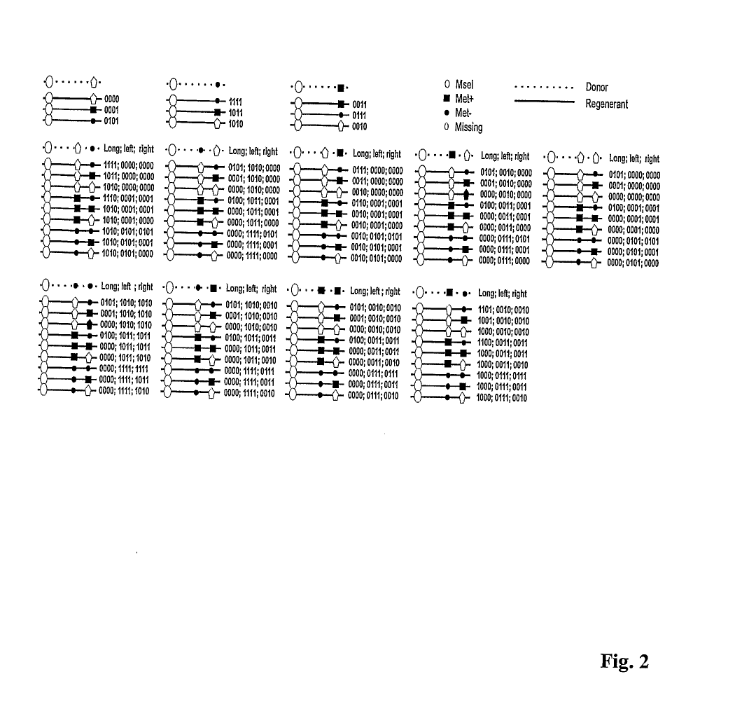

FIG. 2 represents arrangements of the theoretical donor and regenerant genotypes and AFLP patterns putatively amplified by Acc65I/MseI and KpnI/MseI digests. The first and the third number reflect donor and the second and fourth numbers regenerant profiles; the first two numbers depict Acc65I/MseI and the remaining KpnI/MseI digests. 1 and 0 state for presence and absence of a band on a profile.

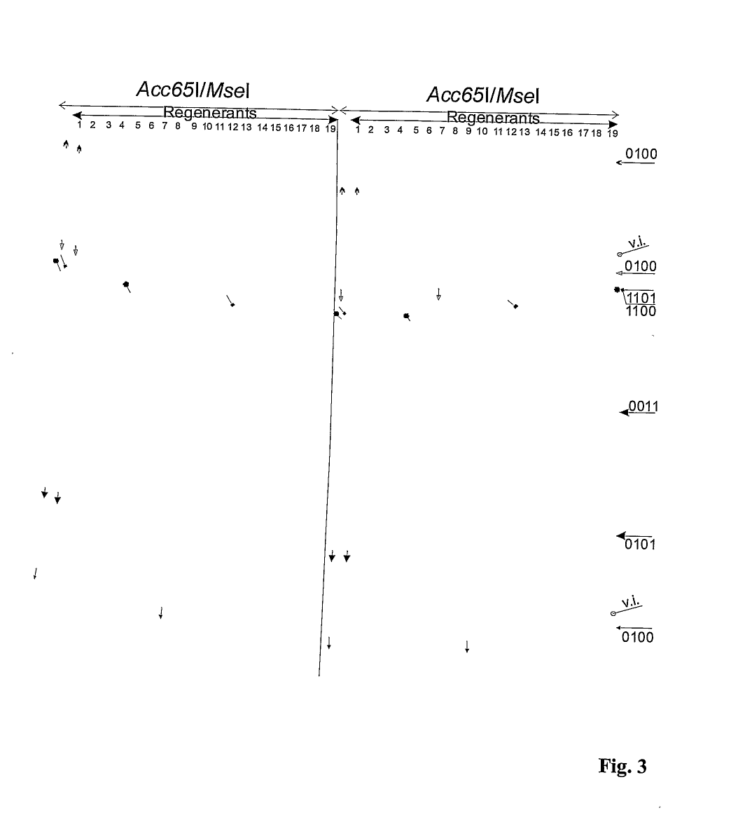

FIG. 3 represents Acc65I/MseI and KpnI/MseI metAFLP fingerprints of the regenerants (lines 1-19) and donor (D) plant, respectively; lanes 1-7—regenerants obtained via embryogenesis; lanes 8-19—regenerants obtained via androgenesis (see FIG. 1); different types of arrows indicate some of the events described as present/absent “1-0” according to the FIG. 7; v.i.—band of variable intensity.

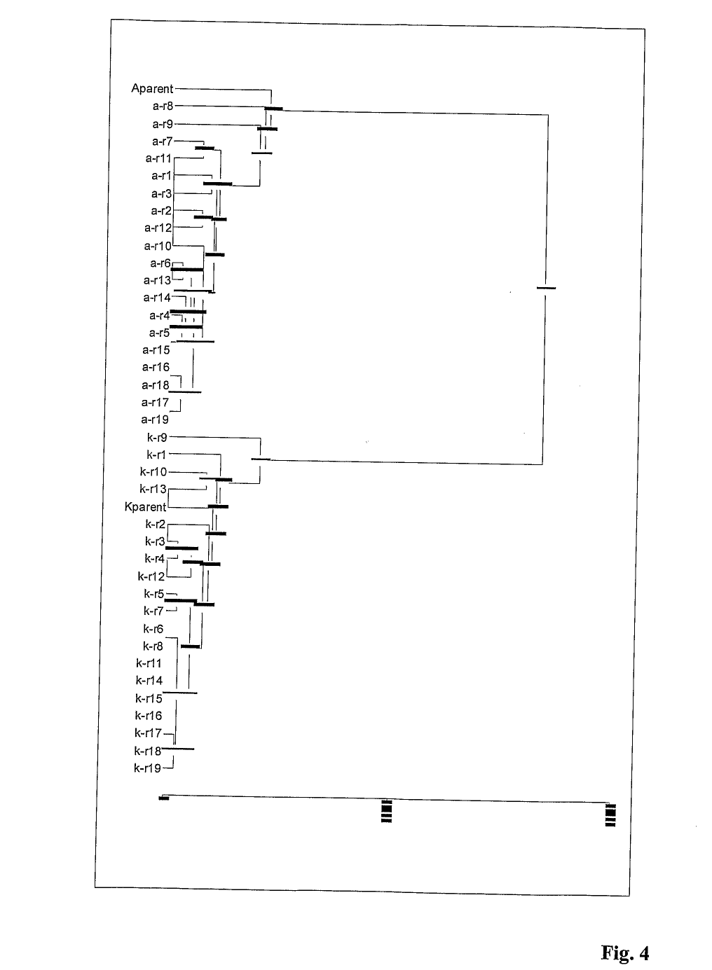

FIG. 4 represents Clustering of the Acc56I/MseI and KpnI/MseI data. ‘a-r’—states for Acc65I/MseI and ‘k-r’ for KpnI/MseI, numbers following ‘a-r’ or ‘k-r’ reflect the given regenerated plant. ‘Aparent’ and ‘Kparent’ state for donor plant.

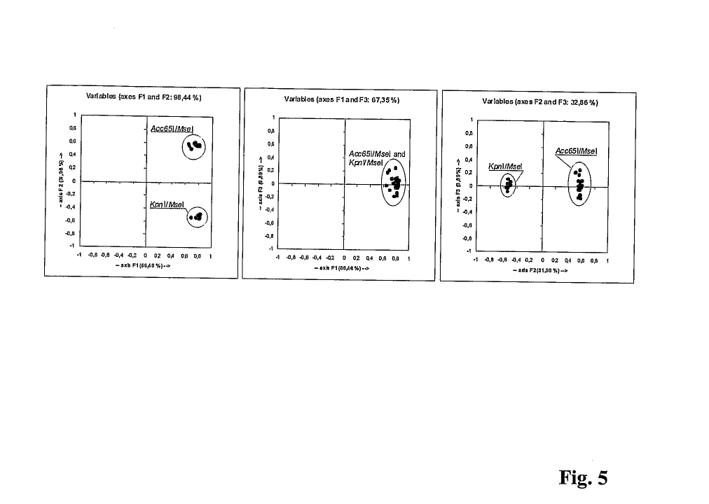

FIG. 5 represents factorial analysis of the fingerprint data generated by Acc65I/MseI and KpnI/MseI digests based on DNAs of the regenerated plants.

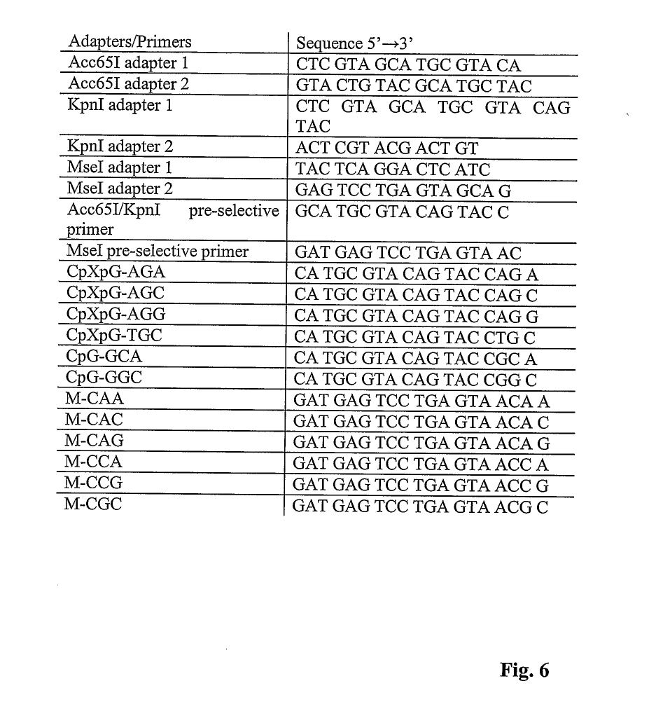

FIG. 6 represents primer sequences for metAFLP.

FIG. 7 represents variants detected by metAFLP. Acc65I/MseI and KpnI/MseI enzyme pairs were used for the digestion of templates; 1/0—AFLP band is present/missing. AFLP pattern column: the first two figures state for Acc65I/MseI and the reminder for KpnI/MseI digests; first and third positions reflect donor and second and fourth regenerant profiles. Column “Event type” classifies the given pattern to the event type. Additionally, the information is given whether the pattern is unequivocal. E, A and S state for embryo-, androgenesis-derived regenerants and all regenerants taken together expressed as absolute and normalized (%) values. Calculations of the tissue culture induced variation are based on event types.

Example embodiments of the present invention defined above are presented below.

### Plant Material

A single double haploid barley plant, obtained via androgenesis from an isolated microspore (Oleszczuk, et al. 2005) was self-fertilised. A set of its sixteen progeny plants that did not differ in phenotypes was grown under controlled conditions (16 h/8 h day/night regime, light intensity 27 μmolm⁻²s⁻¹ and day/night temperatures of, respectively, 10° C. and 12° C.). The set was used to test cryptic segregation due to sexual propagation at the molecular level. After reaching proper developmental stage one progeny plant-donor one (FIG. 1 general scheme) was exploited as a source of immature embryos and anthers. Somatic embryogenesis was induced from immature embryo-derived calli (Zimny and Lörz 1989) grown for three weeks on semi-solid N6 medium (Chu 1978) containing 2 mgl⁻¹ 2,4-D. Somatic embryos were excised from callus, and transferred to a regeneration medium containing 0.4 μM kinetin in order to encourage their germination. Emerging plantlets were transferred to Erlenmeyer flasks until they reached a height of 10-12 cm, after which they were potted, acclimatized for two weeks, and finally grown under standard greenhouse conditions to maturity. To induce androgenesis, tillers of the donor plant were harvested when the microspores were between the uninucleate and the two-nucleate pollen stage. The cut ends of the tillers were held in water under low light intensity for 7 days at 4° C. The spikes were then surface sterilized by dipping first in 70% ethanol and then in 10% sodium hypochloride, after which they were rinsed five times in sterile water. Anthers were excised, placed on semi-solid N6 medium (Chu 1978) supplemented with 2 mgl⁻¹ 2,4-D and 0.5 μM kinetin, and cultured in 6 cm diameter Petri dishes in the dark at 26° C. Embryogenic calli were transferred to 190-2 regeneration medium (Pauk, et al. 1991). Developed androgenic embryos were transferred onto fresh 190-2 medium (Zhuang and Xu 1983) supplemented with hormones according to Pauk (Pauk, et al. 1991) and solidified with 5 g/l Phytagel, and kept under a 16 h/8 h day/night regime. Plantlets were rooted in glass tubes, and later potted, transferred to a growth chamber for a short acclimatization period, and grown to maturity in a controlled environment chamber. Before anthesis, spikes of each regenerant plant were bagged to ensure self-pollination. The two sets of regenerants, obtained via somatic embryogenesis (Set: Regenerants ‘E’), and via androgenesis (Set: Regenerants ‘A’ level 7, FIG. 1), along with that of the donor plant, provided the material to study tissue culture-induced variation.

### DNA Manipulation

Total genomic DNA was isolated from approximately 100 mg of 7-day-old seedling leaf using the DNeasy MiniPrep Kit (Qiagen). metAFLP oligonucleotides were obtained from the Centre for Macromolecular Studies of the Polish Academy of Sciences (L/ódź) (sequences listed in Table 1). The AFLP procedure followed Vos, et al. (Vos, et al. 1995) with minor modifications (Bednarek, et al. 2002), using the restriction enzyme pairs Acc65I/MseI and KpnI/MseI for the initial digestion of genomic DNA. About 500 ng of genomic DNA was digested with Acc65I/MseI and a similar quantity with KpnI/MseI at 37° C. for 3 h, stopping the reaction with an incubation of 15 min incubation at 70° C. The relevant adaptors were ligated to the restriction digest in a final volume of 25 μl at 20° C. for 12 h and diluted 1:3 in TE buffer. Non-selective PCR was performed using a standard amplification profile, in a total volume of 25 μl using pre-selective primers (Table 1), and this reaction was diluted 1:20 in TE. Labelled selective PCRs were carried out in a final volume of 10 μl in the presence of 5′-(³²P) labelled selective primers. Following PCR, samples were denatured by adding 6 μl of 80% formamide loading buffer in the presence of bromophenol blue and xylene cyanol dyes. After a denaturation step (95° C. for 10 minutes followed by quenching at 5° C.), 6 μl of reaction were loaded on a 7% 50 cm×0.4 mm denaturing polyacrylamide gel. After electrophoresis, X-ray films were exposed to gels overnight at −70° C.

### Data Scoring and Analysis

XlStat-pro v.7.5.2 excel add-in software (www.xlstat.com, Addinsoft) was used to perform clustering (UPGMA, Jaccard's coefficient) and factorial (Pearson correlation coefficient without axes rotation) analyses. Estimates of similarity were based on Jaccard's coefficient and clustering was performed by UPGMA (Saiotu and Nei 1986). Analysis of molecular variance (AMOVA) and the Mantel test was carried out using GeneAlex5.1 excel add-in software (Peakall and Smouse 2001). The analytical treatment of the data is explained in the context of the results themselves (see below, Table 2). Kruskal-Wallis statistics (XlStat-pro v.7.5.2) were used to test whether the extent of tissue culture induced variation differed significantly between the embryo- and androgenesis-derived regenerants and among all regenerants.

### Results

### Testing the Uniformity of the Sexually-Derived Progeny

AFLP templates of the sixteen sexually derived plants were generated from both Acc65I/MseI and KpnI/MseI digests, and amplified with ten selective primer combinations (CpG-GGC/MCAA; CpG-GGC/MCAC; CpG-GGC/MCAG; CpXpG-AGA/MCAC; CpXpG-AGA/MCCA; pXpG-AGC/MCAC; CpXpGAGC/MCGA; CpXpG-AGC/MCGC; CpXpG-AGG/MCAA; CpXpG-AGT/MCGC) to produce a total of 764 fragments over both digests. The profiles were invariant across all sixteen plants, except in the cases of selective primer combination (CpG-GGC/MCAC) which delivered two variable Acc65I/MseI fragments, and CpXpG-AGT/MCGC, which produced one variable KpnI/MseI fragment. Thus the background rate of variation is of the order of 0.26% in Acc65I/MseI and 0.13% KpnI/MseI. When recalculated on the basis of the number of events (total number of polymorphic signals that differed from the most representative 0 or 1 signals divided by the number of plants and multiplied by number of the scored AFLPs), we conclude that the sexual mode of reproduction generates polymorphism at a frequency of 0.03 to 0.04%.

### Testing Embryo and Androgenesis-Derived Regenerants

In all, 19 independent viable plants were regenerated from tissue culture. These consisted of seven in set E and twelve in set A. All plants were fully self-fertile and resembled their mother plant in phenotype. Uniform and reproducible fingerprints were generated from the templates of the parental donor and its regenerants using seven selective primer pairs (CpXpGAGG/CAG; CPG-GCA/MCGC; CpXpG-AGC/MCCA; CpXpG-AGG/MCAC; CpGGGC/MCAA; CpXpG-TGC/MCCG; CpXpG-AGA/MCAA) (FIG. 3). A total, 272 fragments were identified across both the Acc65I/MseI and KpnI/MseI AFLP profiles. In each of the sets of profiles, 229 fragments were invariant within the test DNA set (all regenerants and the donor plant). In all, 206 fragments were monomorphic across all samples. Acc65I and KpnI each identified 43 polymorphic fragments, of which 23 were specific to, but monomorphic within one of the isoschizomer combinations. Among these, eight Acc65I/MseI and seven KpnI/MseI fragments varied only with respect to band intensity.

### Statistical Analysis

Cluster analysis demonstrated that the DNAs of the regenerated plants digested with Acc65I/MseI formed clusters that were distinct and independent from those digested with KpnI/MseI. No individual subclusters for sets ‘E’ or ‘A’ were evident (FIG. 4). A factorial analysis (convergence 0.001 after 25 iterations) showed that variables F1 and F2 explain 98% of the variation identified between the Acc65I/MseI and KpnI/MseI sets. The respective proportions for F1-F3 and F2-F3 were 67% and 33%. Once again two clusters were formed, with the KpnI/MseI group being more compact than the Acc65I/MseI one (FIG. 5). AMOVA showed that 69% of the variation resulted from differences between the two digests, but nonetheless the data sets remain highly correlated, as indicated by the Mantel test (R²>0.9991, p=0.001).

Explanation of metAFLP Patterns

The experimental system was designed to detect a number of distinct events that may have occurred during tissue culture. The isoschizomers recognize the same cleavage sequence, but differ in their sensitivity to methylation at the recognition site: Acc65I digests non-methylated, but not methylated sequences, whereas KpnI is insensitive to recognition site methylation. The various changes detectable by this metAFLP approach are summarized in Table 2, in which regenerant profiles are compared to those of the donor. Most (seven) of the patterns could have been generated by a single event, although for some variants more than one explanation is possible. For example, the ‘0011’ pattern describes a fragment that is methylated in both the donor and the regenerant template, so that it is present in the KpnI/MseI, but absent in the Acc56I/MseI digest, and is not a tissue culture-induced event; while the ‘0101’ pattern is delivered where a tissue culture-induced event introduces a non-methylated recognition site not present in the parental template. In contrast, the ‘1000’ pattern could have been generated either from the de-methylation of a single methylated site or the de-methylation of the external site (if two adjacent sites are considered). Some of the patterns could not be assigned to a single event (‘0010’ or ‘0001’) and these are referred to as ‘mixed variation’. All possible donor-regenerant pairs are given in FIG. 2 and permissible classification of the AFLP pattern to the appropriate event type.

### Tissue Culture-Induced Variation

Among the regenerants, 11 out of the 14 possible patterns described in Table 2 were observed at least once. Two mixed (0100 and 1001) and a single sequence variation (0010) patterns were not detected. Across all the regenerants, 94.7% of the restriction fragments were conserved with respect to methylation status, 95.1 and 94.4% were conserved for the embryo- and anther-derived plants. Of these invariant sequences, non-methylated recognition sites were much commoner than methylated ones. Our estimation of tissue culture-induced event frequency was based on the following reasoning: first, the total number of events (5168) was taken as the number of analyzed Acc65I/MseI and KpnI/MseI fragments (272) times the number (19) of regenerants. The equivalent numbers for the E and A regenerants were, respectively, 7×272 (1904) and 12×272 (3264). Distinct tissue culture-induced events were identified by scoring for the presence/absence of fragments in the two digests, across the donor and the regenerant DNAs, as detailed in Table 2 (If the band was missing in one of the digests and present in the second in at least case then it was scored as zero and one, respectively, resulting in two 0-1 matrices of the same dimension). The total number of changes was equal to the whole number events less that of the methylation inheritance (0011 and 1100 patterns). This value equals the number of events comprising total tissue culture-induced variation. The frequency of changes in KpnI/MseI digest was calculated as the total number of differences among donor and regenerant profiles and gives an estimate of sequence variation. Then, the frequency of tissue culture-induced site methylation is the difference between that for total tissue culture-induced variation and that identified in the KpnI/MseI digests. These are converted to percentages by using the appropriate total number of fragments surveyed (5168, 1904 and 3264 for total, E and A). For example, the total number of changes identified based on both digests is 275 (5.3%), the number of sequence changes 101 (2.0%), and the number of methylation variants 174 (3.4%). This last estimate comprises percentages of total tissue culture induced variation (TTCV) less than percentages of sequence variation (SV) calculated based on KpnI/MseI digest. Calculation of the TTCV is the sum of 11 events (Table 2) normalized by the number of events and expressed in percentages. Conserved sites (those that did not change methylation status) were defined in percentage terms as all scored sites less TTCV varied sites (94.7% across all regenerants). Inherited methylation sites were scored as all the sites that did not change methylation status (remaining either methylated or non-methylated). These represent 12.7% of fragments. The frequency of de-methylation is of the same level in somatic and in gametic cells (0.4%). In contrast, de novo methylation occurred a bit less frequently in ‘E’ (1.1%) than in ‘A’ (1.4%). Our estimate of the overall frequencies of methylation events over the two regenerant types is, respectively, 3.0% and 3.6%. ‘Mixed events’ accounted for 1.5% and 2.0%, respectively. Similarly, the frequencies of sequence variation were 1.9% and 2.1%. Finally, the overall proportions of restriction fragments altered as a consequence of the tissue culture treatment were 4.9% and 5.6% for, respectively, the E and A regenerant.

All the regenerated plants displayed either sequence variation or/and changes in methylation pattern, with a mean of about 14 alterations per plant. About 44% of the changes affected the embryogenesis-derived plants. There was little difference in the mean number of sequence changes and alterations in methylation patterns between the embryo- and the anther-derived plants. The tissue culture-induced variation was, however, unevenly distributed among the regenerants, varying from 10 to 16 events in the former, and from 13 to 20 in the latter regenerant types. The Kruskal-Wallis statistics, however, suggest that there is no significant effect of regeneration mode on the extent of tissue culture-induced variation.

### Hotspots of Tissue Culture Induced Variation

We observed eight fragments which appear to have behaved as hotspots of variation, in the sense that all or nearly all of the regenerated plants were altered in sequence or in methylation status in relation to the donor. Thus for example, the Acc65I profile of all regenerants included a fragment that was absent in the donor, while the same fragment was present in the KpnI profiles of both donor and regenerant. The events were classified as mixed (0100—100%; two 1000—100%), de-methylation (0111—100%), de novo methylation (1011—95%) and sequence change and three sequence (0001—100%; 0001—95%; 1010—95%) events.

An important finding is that nearly 95% of sites were faithfully transmitted to all the regenerant plants independently of how they were regenerated (via embryo or androgenesis). Interestingly, only ca. 12% of the sites remain stably methylated which is nearly seven times less than non-methylated ones. This data is comparable with that of (Chakrabarty, et al. 2003) who assessed the extent and pattern of cytosine methylation during somatic embryogenesis in Eleuterococcus senticosus, and showed that over 11% of sites were methylated in embryogenic callus tissue. Rather higher estimates of the extent of methylation have been obtained in rice (16%), and in embryo-derived oil palm (20%) by (Jaligot, et al. 2000).

The strategy we have developed has allowed us to both quantify and qualify the tissue culture-induced variation in barley plants regenerated from embryos and microspores. We show that this occurs at a frequency of two magnitudes higher than the background rate associated with sexual reproduction, and have been able to partition the variation into changes in methylation state and sequence, to estimate the proportion of sites unaffected by variation, to distinguish between methylated and non-methylated sites, and to predict the appearance of bands with varying intensity. We may also examine changes that affected individual plants and specify the degree they may differ from the donor one. Although not attempted here we believe that by using specially designed selective primers it should be possible to differentiate among various types of methylation and thereby obtain some insight into sequence-variation defining whether it is induced by migration of mobile elements, by changes in microsatellite copy number or by mutations within functional genomic regions belonging to different protein families. We believe that our analytical approach is the first which can differentiate between the two major classes of tissue-induced epigenetic (methylation) and genetic (sequence mutation) variation, and provide a quantitative and qualitative description of their relative contribution. This represents a means to access tissue-induced variation among regenerants sharing a uniform phenotype. Moreover, after minor modifications the approach could be used to study inheritance of the tissue culture induced variation among progenies of the regenerants giving characteristics of somaclonal variation. It is possible to anticipate the development of this approach for the dissection of epigenetic inheritance.

### REFERENCES

Aina R, Sgorbati S, Santagostino A, Labra M, Ghiani A, Citterio S. (2004) Specific hypomethylation of DNA is induced by heavy metals in white clover and industrial hemp. Physiol Plant 121:472-480.
Arene L, Pellegrino C, Gudin S. (1993) A comparison of the somaclonal variation level of Rosa hybrida L. cv. Meirutral plants regenerated from callus or direct induction from different vegetative and embryonic tissues. Euphitica 71:83-90.
Bednarek P T, Kubicka H, Zawada M. (2002) Morphology, cytology and BSA based testing on limited segregation population AFLPs. Cell Mol Biol Let 7:635-648.
Bouman H, De Klerk G-J. (2001) Measurement of the extent of somaclonal variation in begonia plants regenerated under various conditions. Comparison of three assays. Theor Appl Genet 102:111-117.
Brar D S, Jain S M. (1998) Somaclonal variation: mechanism and applications in crop improvement. In: Somaclonal variationa and Induced Mutations in Crop Improvement. Kluwer Academic Publishers, Dordrecht, Boston, London: 16-37.
Bregitzer P, Halbert S E, Lemaux P G. (1998) Somaclonal variation in the progeny of transgenic barley. Theor Appl Genet 96:421-425.
Breyne P, Rombaut D, Gysel A, Montagu M, Gerats T. (1999) AFLP analysis of genetic diversity within and between Arabidopsis thaliana ecotypes. Mol Gen Genet 261:627-634.
Brown P T H, Lange F D, Kranz E, Lörz H. (1993) Analysis of single protoplasts and regenerated plants by PCR and RAPD technology. Molecular General Genetics 237:311-317.
Cervera M, Ruiz-Garcia L, Martinez-Zapater J. (2002) Analysis of DNA methylation in Arabidopsis thaliana based on methylation-sensitive AFLP markers. Molecular Genetics and Genomics 268:DOI 10.1007/s00438-002-0772-4.
Chakrabarty D, Yu K W, Paek K Y. (2003) Detection of DNA methylation changes during somatic embryogenesis of Siberian ginseng (Eleuterococcus senticosus). Plant Science 165:61-68.
Chu C. (1978) The N6 medium and its applications to anther culture of cereal crops. Proc. Symp. Plant Tissue Culture, Science Press Beijing: 43-50.
Devaux P, Kilian A, Kleinhofs A. (1993) Anther culture and Hordeum bulbosum-derived barley doubled haploids: mutations and methylation. Mol. Gen. Genet. 241:674-679.
Donini P, Elias M L, Bougourd S M, Koebner R M D. (1997) AFLP fingerprinting reveals pattern differences between template DNA extracted from different plant organs. Genome 40:521-526.
Fourré J-L, Berger P, Niquet L, André P. (1997) Somatic embryogenesis and somaclonal variation in Norway spruce: morphogenetic, cytogenetic and molecular approaches. Theor Appl Genet 94:159-169.
Freyssinet M, Freyssinet G. (1988) Fertil plant regeneration from sunflower (Heliantus annuus L.) immature embryos. Plant Sci. 56:177-181.
Gaj M D. (2001) Direct somatic embryogenesis as a rapid and efficient system for in vitro regeneration of Arabidopsis thaliana. Plant Cell Tiss Org 64:39-46.
Gray D J, Compton M E, Harrell R C, Contliffe D J. (1995) Somatic embryogenesis and the technology of synthetic seed. In: Biotechnology in Agriculture and Forestry. Somatic Embryogenesis and synthetic seed I. Springer, Berlin 30:126-151.
Guzy-Wróblewska J, Szarejko I. (2003) Molecular and agronomic evaluation of wheat double haploid lines obtained through maize pollination and anther methods. Plant Breeding 122:305-313.
Habu Y, Kakutani, T. and Paszkowski, J. (2001) Epigenetic developmental mechanisms in plants: Molecules and targets of plant epigenetic regulation. Curr. Opin. Genet. Dev. 11:215-220.
Hossain M A, Konisho K, Minami M, Nemoto K. (2003) Somaclonal variation of regenerated plants in chili pepper (Capsicum annuum L.). Euphytica 130:233-239.
Jacobsen S E, Sakai, H., Finnegan, E. J., Cao, X. and Meyerowitz, E. M. (2000) Ectopic hypermethylation of flower-specific genes in Arabidopsis. Curr. Biol. 10:179-186.
Jaligot E, Beulé T, Rival A. (2002) Methylation-sensitive RFLPs: characterisation of two oil palm markers showing somaclonal variation-associated polymorphism. Theor Appl Genet 104:1263-1269.
Jaligot E, Rival A, Beulé T, Dussert S, Verdeil J-L. (2000) Somaclonal variation in oil palm (Elaeis guineensis Jacq.): the DNA methylation hypothesis. Plant Cell Reports 19:684-690.
Kakutani T, Munakata, K., Richards, E. J. and Hirochika, H. (1999) Meiotically and mitotically stable inheritance of DNA hypomethylation induced by ddm1 mutation of Arabidopsis thaliana. Genetics 151:831-838.
Kudirka D T, Schaeffer G W, Beanzinger P S. (1989) Stability of ploidy in meristems of plants regenerated from anther calli of wheat (Triticum aestivum L. em. Thell.). Genome 32:1068-1073.
Larkin P J, Scowcroft W R. (1981) Somaclonal variation—a novel source of variability from cell cultures for plant improvement. Theor Appl Genet 60:197-214.
Linacero R, Freitas A E, Vázquez A M. (2000) Hot spots of DNA instability revealed through the study of somaclonal variation in rye. Theor Appl Genet 100:506-511.
Munthali M T, Newbury H J, Ford-Lloyd BV. (1996) The detection of somaclonal variants of beet using RAPD. Plant Cell Reports 15:474-478.
Oleszczuk S, Sowa S, Zimny J. (2005) Androgenic response to pre-culture stress in the microspore cultures of barley. Protoplasma in press
Pauk J, Manninen O, Mattila I, Salo Y, Pulli S. (1991) Androgenesis in hexaploid spring wheat F2 populations and their parents using a multiple-step regeneration system. Plant Breeding 107:18-27.
Peakall R, Smouse P E. (2001) GenALEx V5: Genetic Analysis in Excel. Population genetic software for teaching and research. Australian National University, Canberra. http://www.anu.edu.au/BoZo/GenAlEx/
Peraza-Echeverria S, Herrera-Valencia V A, James-Kay A. (2001) Detection of DNA methylation changes in micropropagated banana plants using methylation-sensitive amplification polymorphism (MSAP). Plant Science 161:359-367.
Peshke V M, Phillips R L. (1991) Activation of the maize transposable element Suppreassor-mutator (Spm) in tissue culture. Theor Appl Genet 81:90-97.
Portis E, Acquadro A, Comino C, Lanteri S. (2003) Analysis of DNA methylation during germination of pepper (Capsicum annuum L.) seeds using methylation-sensitive amplification polymorphism (MSAP). Plant Science 166:169-178.
Remotti P C. (1998) Somaclonal variation and in vitro selection for crop improvement. In: Somaclonal Variation and Induced Mutations in Crop Improvement. Eds. Jain, S. M., Brar, D. S., Ahloowalia, B. S.: 169-201.
Reyna-López G E, Simpson J, Ruiz-Herrera J. (1997) Differences in DNA methylation patterns are detectable during the dimorphic transition of fungi by amplification of restriction polymorphisms. Mol Gen Genet 253:703-710.
Roseland C S, Espinasse A, Grosz T J. (1991) Somaclonal variats of sunflower with modified coumarin expression under stress. Euphitica 54
Saiotu N, Nei M. (1986) The number of nucleotides required to determine the branching order of three species, with special reference to human-Chimpanzee-Gorilla divergence. J. Mol. Evaluation 24:189-204.
Thomas E, Bright S W J, Franklin J, Lancaster V, Miflin B J. (1982) Variation amongst protoplast-derived potato plants (Solanum tuberosum cv, “Maris Bar”). Theor Appl Genet 62:65-68.
Vendrame W A, Kochert G, Wetzstein H Y. (1999) AFLP analysis of variation in pecan somatic embryos. Plant Cell Reports 18:853-857.
Vos P, Hogers R, Bleeker M, Lee T, Homes M, Frijters A, Pot J, Peleman J, Kuiper M, Zauber M. (1995) AFLP: a new technique for DNA fingerprinting. Nucl Acids Res 23:4407-4414.
Wang Z Y, Nagel J, Potrykus I, Spangenberg G. (1993) Plants from cell suspension-derived protoplasts in Lolium species. Plant Sci 94:179-193.
Xiong L Z, Xu C G, Saghai Maroof M A, Zhang Q. (1999) Patterns of cytosine methylation in an elite rice hybrid and its parental lines, detected by a methylation-sensitive amplification polymorphism technique. Mol Gen Genet 261:439-446.
Zhuang J J, Xu J. (1983) Increasing differentiation frequencies in wheat pollen calus. In: Cell and Tissue Culture Techniques for Cereal Crop Improvement (Hu, H. and Vega, M. R. Eds). Science Press Beijing 431
Zimny J, Lörz H. (1989) High frequency of somatic embryogenesis and plant regeneration of rye (Secale cereale L.). Z. Pflanzenzüchtung 102:89 100.

1. A method for the identification of variability induced by the derivation method in in vitro cultures at the level of regenerant plants, as well as variation inherited as a result of generative propagation, characterised in that the method consists of the following stages:
1) preparation of a single plant, a doubled haploid, derived via direct embryogenesis from a single microspore and/or egg cell to be used in the production of donor plants through self-pollination, which are to be a control for the level of generative reproduction-caused genetic and epigenetic variability; wherein the donor plants constitute a source of explants used for in vitro cultures, where each donor plant is an ancestor of a separate set of regenerants, and each regenerant is used to obtain a set of plants which are the progeny of a given regenerant;
2) the production of regenerants derived through somatic embryogenesis, pollen androgenesis and isolated microspores, or chromosome elimination, henceforth called methods of derivation, where the regenerants serve to estimate genetic and epigenetic changes induced directly by their method of derivation;
3) production of generative progeny of regenerants, which is derived via somatic embryogenesis, pistil androgenesis and isolated microspores or chromosome elimination, wherein this progeny along with the donor plant constitute the research material for the analysis of variability inherited generatively, henceforth called somaclonal variability;
4) in donor plants and an individual plant which are controls of generative propagation, estimation of genetic and epigenetic variability induced during the generative propagation of an individual plant in two AFLP systems, using isoschizomers both sensitive and insensitive to methylation at the restriction-site and/or adjacent areas, and a representative number of pairs of selective primers identical in all AFLP analyses, where:
a) the estimation of genetic and epigenetic variability induced during the generative propagation of an individual plant in two AFLP systems among donor plants is alternatively conducted without an individual plant, but with the use of isoschizomers both sensitive and insensitive to methylation at the restriction-site and/or adjacent areas as well as a representative number of pairs of selective primers identical in all AFLP analyses;
b) the estimation of genetic and epigenetic variability induced during the generative propagation of an individual plant in two AFLP systems among donor plants is alternatively conducted without an individual plant, but with the use of isoschizomers both sensitive and insensitive to methylation at the restriction-site and/or adjacent areas as well as a representative number of pairs of selective primers different from those used in all AFLP analyses;
5) estimation of the repeatability of the two AFLP technique variants: repeatability control of AFLP based on the identity of several DNA samples of the same plant through the use of AFLP and isoschizomers both sensitive and insensitive to methylation at the restriction-site and/or adjacent areas as well as a representative number of pairs of selective primers identical in all AFLP analyses;
6) estimation of the repeatability of the two AFLP technique variants: repeatability control of AFLP based on the identity of several DNA samples of the same plant through the use of AFLP and isoschizomers both sensitive and insensitive to methylation at the restriction-site and/or adjacent areas as well as a representative number of pairs of selective primers using a different set of selective AFLP primers than those used in the other AFLP analyses;
7) identification of variability induced by the method of derivation of the regenerants in relation to the donor plant is performed in two AFLP variants using isoschizomers both sensitive and insensitive to methylation at the restriction-site and/or adjacent areas as well as a representative number of pairs of selective primers identical in all AFLP analyses;
8) the identification of the tissue culture induced variability among regenerants in relation to the donor plant is performed in two AFLP technique variants utilising isoschizomers both sensitive and insensitive to methylation at the restriction-site and/or adjacent areas as well as a representative number of pairs of selective primers identical in all AFLP analyses;
9) the identification of somaclonal variability among regenerant progeny in relation to the donor plant is carried out using two AFLP technique variants utilising isoschizomers both sensitive and insensitive to methylation at the restriction-site and/or adjacent areas as well as a representative number of pairs of selective primers identical in all AFLP analyses;
10) the molecular analysis of the experimental material using the AFLP technique taking into account isoschizomers both sensitive and insensitive to methylation at the restriction-site and/or adjacent areas as well as a representative number of pairs of selective primers identical in all AFLP analyses is comprised of the following stages:
a) analyses of AFLP patterns of donor plants with respect to genetic and epigenetic variability induced during the generative propagation of an individual plant, a control of generative propagation,
b) analyses of AFLP patterns for the control of the reproducibility of both AFLP technique variants,
c) analysis of AFLP patterns for regenerants in comparison to donor plants or separate regenerants in relation to appropriate donor plants;
d) analyses of AFLP patterns for donor plant, its regenerant and regenerant progeny, or distinct regenerant progeny lines along with appropriate donor and its regenerant plants;
11) identification of the 14 types of events recognized at the binary matrix level for systems sensitive and insensitive to methylation, which are the result of a comparison between the donor plant with regenerants and between donor plant with regenerant progeny simultaneously in two AFLP systems, wherein the identification of event types is carried out individually for each regenerant and as bulk for regenerant progenies and is represented as 14 binary matrices which describe each of the events independently, subdivided into methods of derivation; wherein the columns represent consecutive regenerants or bulks and rows represent consecutive AFLP fragments;
12) assignment of certain event types to a given type of variability induced the method of derivation in relation to each regenerant;
13) identification of the 14 types of events recognized at the binary matrix level for systems sensitive and insensitive to methylation, which are the result of a comparison between the donor plant with regenerant simultaneously in two AFLP systems;
14) identification of the 14 types of events recognized at the binary matrix level for systems sensitive and insensitive to methylation, which are the result of a comparison between the donor plant with regenerant progeny simultaneously in two AFLP systems, where the identification of event types is performed through the summation of the genotypes of a given summary progeny, and through the comparison of such a total AFLP profile of regenerant progeny with the donor plant profile,
15) the bulk regenerant progeny genotype expressed via an AFLP profile for systems sensitive and insensitive to methylation at the level of individual AFLP fragments has a different value from that of the donor plant in a given AFLP system, so long as a given fragment within it is polymorphic, or is totally different from this value for the donor plant; in the opposite case the values for the donor plant and bulk regenerant progeny for a given AFLP fragment in a given AFLP system are identical,
16) production of a quantitative matrix for regenerants, where the columns contain data for consecutive regenerants grouped by the method of derivation, and the rows contain the 14 types of events which may be assigned to each regenerant progeny;
17) production of a quantitative matrix for regenerant progeny, where the columns contain bulk data for regenerant progeny grouped by the method of derivation, and the rows contain the 14 types of events which may be assigned to each bulk regenerant progeny;
18) production of a quantitative matrix for events in regenerants, where this is a quantitative matrix for post-transposition regenerants, where the columns contain 14 events and the rows contain regenerants grouped by the method of derivation;
19) calculation of the total number of events based on the binary matrix for individual methods of derivation of regenerants as well as for regenerants, where the total number of events is calculated as a product of the number of AFLP fragments identified in both AFLP systems for all pairs of selective primers multiplied by the number of regenerants derived by a given method;
20) calculation of the total number of events based on the binary matrix for individual methods of derivation of regenerants as well as for regenerant progenies in bulk, where the total number of events corresponds to the number of DNA fragments identical or different in both AFLP systems for all pairs of selective primers used in the analysis;
21) calculation of the error stemming from generative propagation is carried out based on the binary matrix of donor plants and a single plant; determination of the number of events different from those in a single plant belonging to the donor plants, and the calculation of the total number of events for the donor plants;
22) calculation of the error stemming from the AFLP technique is carried out based on the binary matrix for DNA samples obtained from the same plant and subjected to the stages of the AFLP technique using isoschizomers sensitive and insensitive to methylation at the restriction site or its immediate surroundings as well as a representative number of selective starter pairs identical in all AFLP systems, where the total number of events and the number of polymorphic events are calculated, and the latter number is expressed as a percentage;
23) determination, based on binary matrices for regenerants and the donor plant, of groups of events describing: (a) sequence variation connected with any changes in DNA nucleotide sequences based on AFLP data from the system insensitive to methylation and (b) epigenetic variability, the de novo variability of methylation patterns, and additionally mixed variation which is sequence and methylation variability based on data for both AFLP systems;
24) determination, based on binary matrices for bulk regenerant progeny and the donor plant, of event groups describing (a) sequence variation connected with all DNA nucleotide changes based on AFLP data from the system insensitive to methylation and (b) epigenetic variability, the de novo variability of methylation patterns, and additionally mixed variation which is sequence and methylation variability, and (c) inheritance of methylation patterns as related to the presence and/or lack of methylation at the restriction site or its vicinity, based on data for both AFLP systems;
25) identification of events particularly subject to the activity of factors inducing variability in in vitro cultures;
26) sequence analysis of individual event types and/or broad sequence variability, where variability is identified using AFLP technique variants using primers directed against conservative sequences within mobile elements, repeating sequences or the sequences of conservative gene families;
27) statistical analysis of the donor plant and its regenerants as well as regenerant progeny, and possibly distinct regenerants and regenerant progenies along with appropriate donor plants, as well as AFLP reproducibility control and generative propagation control:
a) statistical analysis of the results of AFLP using parametric analysis, Mantel's test and/or Cronbach's alpha coefficient as well as molecular variance analysis using the Fₛₜ or Phiₛₜ coefficients;
b) determination of the error of the tissue culture induced and somaclonal variability estimates, based on the Mantel test correlation coefficient;
c) determination of the quantitative parameters of variability induced by the method of derivation of regenerants and somaclonal variability for regenerants, individual regenerant progenies, taking into account the method of derivation and giving their statistical significance level;
d) statistic determination of differences between individual types of events induced in in vitro cultures for regenerants, as well as bulk regenerant progeny, grouped by method of derivation and supplemented by their statistical significance using quantitative matrices of events;
e) statistical analysis of the comparison between variability induced by the method of derivation as well as somaclonal variation, determined through the comparison of the donor plant profile, and the bulk profile of regenerant progeny and their types: sequence variability and/or broad sequence variability, de novo methylation, demethylation, mixed variability, inheritance of: methylation patterns, non-methylated sites or conservative sites in regenerant progeny, grouped by method of derivation as well as a comparison by method of derivation of regenerant progeny, where quantitative matrices are used for events;
f) statistical comparisons of regenerants, and the bulk regenerant progenies for different methods of derivation for a given regenerant progeny, where quantitative matrices of the progeny are used;
g) statistical comparison of the results of the above mentioned points for individual regenerants and bulk regenerant progenies amongst one another;
h) statistical or qualitative comparison of somaclonal variation between individual plants being the generative progeny of regenerants;
i) collation of the existing results in the form of a compilation indicating quantitative characteristics of variability induced by the method of derivation determined at the regenerant level, as well as quantitative characteristics of inherited variability determined at the level of regenerant progeny.

2. A method according to claim 1, characterised in that the identification of the 14 types of events recognized at the level of binary matrix is selected from among the events represented in FIG. 7.

3. A method according to claim 1, characterised in that to derive donor plants, a single plant is used which is a doubled haploid derived from an isolated microspore, wherein it is treated as a control of the genetic and epigenetic variability of donor plants, and wherein genetic and epigenetic variability of generatively propagated material, donor plants, is determined based on AFLP profiles obtained from restriction endonuclease isoschizomers recognizing and digesting identical sequences, but differing in their sensitivity to methylation at or about the restriction site, wherein one of the isoschizomers is insensitive to methylation, and wherein in a system sensitive and insensitive to methylation no differences are observed in the AFLP profiles of donor plants, then the level of variability induced through generative propagation and identified with a given pair of selective primers is insignificant and when in a system sensitive and insensitive to methylation there are differences in AFLP profiles for donor plants between a system sensitive and a system insensitive to methylation, a percentage portion of such an event is determined, along with the background level, generative propagation error, and when there are differences in AFLP profiles for experimental replicants on the same DNA samples, the AFLP technique error is estimated in order to determine experimental error.

4-7. (canceled)

8. A method according to claim 1, characterised in that the integral portion of the research material is composed of donor plants, along with a single plant, donor plant regenerants and separate regenerant progenies and the donor plant along with regenerants and regenerant progeny compose the core of research material for the study of variability induced by the method of derivation or somaclonal variability, respectively.

9. (canceled)

10. A method according to claim 1, characterised in that the molecular analysis is performed using the AFLP technique in two variants, using isoschizomers differing in their sensitivity to methylation and one of the isoschizomers is completely insensitive to methylation.

11. (canceled)

12. A method according to claim 1, characterised in that the AFLP technique is composed of the following stages: isolation of genomic DNA, digestion of the genomic DNA with one pair of restriction endonucleases and digestion of another portion of the same DNA with another pair of restriction endonucleases, annealing of synthetic heteroduplexes to the free restriction ends, initial amplification of fragments obtained in such a manner by way of primary PCR, selective amplification, separation of the PCR products in a polyacrylamide gel, visual profile analysis and wherein in the identification of segregated PCR products, and isotope marker is used which is introduced to the 5′ end of the primer complementary to the adaptor annealed to the restriction site freed by the isoschizomers or wherein other methods of visualising AFLP fragments are used, including fluorescence.

13-14. (canceled)

15. A method according to claim 1, characterised in that the profiles of both AFLP variants for the donor plant along with its regenerants and taking into account the method of derivation and AFLP reproducibility control are performed on identical AFLP primer pairs.

16. A method according to claim 1, characterised in that AFLP profile analysis and their conversion to binary matrices is based on the fact that the AFLP profiles of both restrictase systems for all pairs of selective primers are entered as a binary matrix into a spreadsheet, where the data set obtained for one pair of selective AFLP primers is placed below the previous one such that each column represents a total binary profile for a donor plant in one AFLP system, and the subsequent columns contain analogous data for regenerants and controls or data for regenerant progenies and controls, wherein data for the second AFLP system is recorded in subsequent columns, wherein the bands from one system are ordered like those from the previous system (in rows), and where a given band does not appear a zero is entered in the appropriate row of the spreadsheet, and wherein the AFLP profile for regenerant progeny is represented in the form of a bulk genotype per AFLP variant, sensitive and insensitive to methylation separately.

17-18. (canceled)

19. A method according to claim 1, characterised in that the generative propagation control is carried out through the summation of the number of signals (events) from a single plant, where the number of changes is calculated as a percentage of the total number of events.

20. A method according to claim 1, characterised in that the parametric analysis, Mantel's test correlation analysis and molecular variability analysis are performed using binary matrices of AFLP systems sensitive and insensitive to methylation for regenerants when the variability induced in culture is being analyzed, and wherein the factorial analysis is used as the graphical representation of the AFLP results.

21. (canceled)

22. A method according to claim 1, characterised in that the identification of the 14 types of events recognized at the level of AFLP pattern and assigned to variability induced by the method of derivation is performed on the basis of AFLP patterns, wherein the donor plant profile is used along with regenerant profiles in both systems, and subsequently the profile of each regenerant is compared to the donor plant profile in order to identify these events or assigned to somaclonal variability is carried out on the basis of AFLP patterns, where the donor plant profile is used along with profiles in both AFLP systems, and the bulk profile of regenerant progeny is compared to the donor plant profile in order to identify event types, wherein it is assumed that in case of polymorphic AFLP fragments of the bulk regenerant progeny genotype, a value different than that of the donor plant is entered or a different value if it is different from that of the donor plant in all regenerant progenies for a given AFLP system.

23. (canceled)

24. A method according to claim 1, characterised in that when the 14 event types comprise the basis for their segregation into groups: sequence variability, methylation variability (methylation and demethylation), mixed variability and inheritance of methylation patterns, wherein it is taken into account that sequence variability is unequivocally confirmed in a system insensitive to methylation.

25. A method according to claim 1, characterised in that the analysis of the significance of individual variability types and events within regenerants and regenerant progeny plants, as grouped by the method of derivation, is performed based on qualitative matrices and quantitative matrices of events describing all theoretically possible events, as well as regenerant plants and regenerant progeny, and the statistical analysis results for individual regenerants and bulk regenerant progenies obtained through various means are compiled into a whole; quantitative parameters are entered for variability induced by the method of derivation and of somaclonal variability for individual regenerants and bulk regenerant progenies as well as for separate regenerant progenies grouped by the method of derivation and event types along with statistical parameters, and those event types are identified, which are rare in culture or which do not occur at all, as well as those which are particularly subject to the activity of factors inducing variability at the level of regenerant and regenerant progeny, as well as to determine their quantitative parameters, and wherein individual event types are studied at the molecular level through the sequence analysis of AFLP fragments representing individual types of events, and by the same token types of somaclonal variability.

26-28. (canceled)

29. A method according to claim 1, characterised in that one or more bulk regenerant progeny is analyzed and the quantitative characteristics of an event are given for each individual regenerant progeny: sequence variability, broad sequence variability, epigenetic variability of methylation patterns (de novo methylation, demethylation), stability of inherited methylated and non-methylated sites (conservative), against changes induced in cultures as well as entering quantitative differences in variability between different derivation methods of plant material and wherein statistical analysis is performed of the significance of individual event types among the regenerants, bulk regenerant progeny and individual regenerant progenies as needed, grouped by the method of derivation of the research material.

30-31. (canceled)

32. A method according to claim 1, characterised in that the somaclonal variability level of individual plants which are regenerant progeny is determined independently, using the principles used for somaclonal variability.

33. A method according to claim 1, characterised in that the hypotheses relating to the sources of variability induced by the method of derivation of regenerants and that inherited as a result of generative propagation are tested and the detailed analysis is performed of the causes of sequence variability after introducing minor AFLP procedural changes, based on the selective amplification of research material using a tagged primer complementary to conservative sites of transposable elements, or to macrosatellite sequences.

34. (canceled)

35. A method according to claim 1, characterised in that the segregation of genetic and epigenetic markers are analyzed for mapping populations derived through plant-plant crosses which are the progeny of regenerants with a known genotype, based on AFLP profiles for both AFLP systems.

36. A method according to claim 1, characterised in that the method is designed for deriving plants in vitro with a lowered or enhanced level of somaclonal variability through the identification of the causes of this variability.

37. A method according to claim 1, characterised in that the genetic and epigenetic purity of arbitrary material derived through in vitro culture is verified.

38. A method according to claim 1, characterised in that when the method is used in the analysis of the inheritance of epigenetic variability during sexual crossing and/or the method is a tool for the identification of epigenetic markers linked with the utilitarian characteristics of the plant material and/or wherein the method is used for the identification of differences in DNA methylation patterns for various tissues of an individual plant.

39-40. (canceled)


SEQUENCE LISTING (20 sequences)

SEQ ID NO: 1 - DNA, 17 bp, Artificial
  Acc65I adapter 1
ctcgtagcatgcgtaca

SEQ ID NO: 2 - DNA, 18 bp, Artificial
  Acc65I adapter 2
gtactgtacgcatgctac

SEQ ID NO: 3 - DNA, 21 bp, Artificial
  KpnI adapter 1
ctcgtagcatgcgtacagtac

SEQ ID NO: 4 - DNA, 14 bp, Artificial
  KpnI adapter 2
actcgtacgactgt

SEQ ID NO: 5 - DNA, 15 bp, Artificial
  MseI adapter 1
tactcaggactcatc

SEQ ID NO: 6 - DNA, 16 bp, Artificial
  MseI adapter 2
gagtcctgagtagcag

SEQ ID NO: 7 - DNA, 16 bp, Artificial
  Acc65I/KpnI pre-selective primer
gcatgcgtacagtacc

SEQ ID NO: 8 - DNA, 17 bp, Artificial
  MseI pre-selective primer
gatgagtcctgagtaac

SEQ ID NO: 9 - DNA, 18 bp, Artificial
  CpXpG-AGA
catgcgtacagtaccaga

SEQ ID NO: 10 - DNA, 18 bp, Artificial
  CpXpG-AGC
catgcgtacagtaccagc

SEQ ID NO: 11 - DNA, 18 bp, Artificial
  CpXpG-AGG
catgcgtacagtaccagg

SEQ ID NO: 12 - DNA, 18 bp, Artificial
  CpXpG-TGC
catgcgtacagtacctgc

SEQ ID NO: 13 - DNA, 18 bp, Artificial
  CpG-GCA
catgcgtacagtaccgca

SEQ ID NO: 14 - DNA, 18 bp, Artificial
  CpG-GGC
catgcgtacagtaccggc

SEQ ID NO: 15 - DNA, 19 bp, Artificial
  M-CAA
gatgagtcctgagtaacaa

SEQ ID NO: 16 - DNA, 19 bp, Artificial
  M-CAC
gatgagtcctgagtaacac

SEQ ID NO: 17 - DNA, 19 bp, Artificial
  M-CAG
gatgagtcctgagtaacag

SEQ ID NO: 18 - DNA, 19 bp, Artificial
  M-CCA
gatgagtcctgagtaacca

SEQ ID NO: 19 - DNA, 19 bp, Artificial
  M-CCG
gatgagtcctgagtaaccg

SEQ ID NO: 20 - DNA, 19 bp, Artificial
  M-CGC
gatgagtcctgagtaacgc